(19) United States
(12) Patent Application Publication
(10) Pub. No.: US 2010/0297268 A1
(43) Pub. Date: Nov. 25, 2010

(54) AGENT FOR INCREASING BLOOD ADIPONECTIN QUANTITY

(75) Inventor: Fumitaka Ueda, Kanagawa (JP)

Correspondence Address: SUGHRUE-265550, 2100 PENNSYLVANIA AVE. NW, WASHINGTON, DC, 20037-3213, US

(73) Assignee: FUJIFILM CORPORATION, Tokyo (JP)

(21) Appl. No.: 12/864,361

(22) PCT Filed: Jan. 22, 2009
(86) PCT No.: PCT/JP2009/051397

§ 371 Date: Jul. 23, 2010

(30) Foreign Application Priority Data
Jan. 23, 2008 (JP) 2008-012733

(51) Int. Cl. A61K 36/37; A61P 3/10; A61P 3/04

(52) U.S. Cl. 424/725

(57) ABSTRACT

An agent for increasing blood adiponectin quantity is provided, the agent including: a pulverized product or extract of a plant of the genus Salacia.

### TECHNICAL FIELD

This invention relates to an agent for increasing blood adiponectin quantity, which comprises a pulverized product or extract of a plant of the genus Salacia and a food or medicine containing its components.

### BACKGROUND ART

The root and trunk of a plant of the genus Salacia have been used as a natural drug by a traditional medical science, aayurveda, in India and Sri Lanka. It has been handed down in Sri Lanka that the root skin of Salacia reticulata is effective in treating rheumatism, gonorrhea and a skin disease and is also used in the treatment of initial stage diabetes mellitus. In India, the root of Salacia oblonga is used in similar treatments and it is said that Salacia chinensis is also used in the treatment of diabetes mellitus, and it has been reported that its action mechanism is the sugar absorption suppressing action based on the α-glucosidase activity inhibition (FOOD Style 21, vol. 6, no. 5, pp. 72-78).

Though most of the effects of Salacia so far reported are as described in the above, this time, we have conducted intensive studies on the influence of ingestion of Salacia on blood and found as a result that ingestion of Salacia significantly increases adiponectin concentration in blood.

Adiponectin is a fat tissue-specific secretory protein isolated in 1996 from a human fat tissue (Maeda K et al., Biochem. Biophys. Res. Commun., 221: 286 (1996)) and is present in not only fat tissue but blood in a large amount (from 3 to 10 μg/ml in normal person) (Arita Y et al., Biochem. Biophys. Res. Commun., 257: 79-83 (1999)). Since various actions, such as improvement of arteriosclerosis, improvement of hypertension, improvement of lipid metabolism, improvement of insulin sensitivity, anti-inflammatory action, inhibition of hepatic fibrosis and the like, have been found in adiponectin (Japanese Patent No. 3018186, JP-A-2000-256208, US Patent Publication No. 2002/0132773 and JP-A-2005-325072), it can be said that a medicine or food or drink which can increase adiponectin concentration in blood has an action to prevent or improve metabolic syndrome.

Though there are already known medicines which increase adiponectin concentration, these cannot be used easily because of the problems of safety and side effects.

Accordingly, under the current state that anti-metabolic syndrome countermeasure is becoming social problem, concern has been directed toward the development a safe substance having the action to accelerate and reinforce production of adiponectin in the living body.

### DISCLOSURE OF THE INVENTION

The problem that the invention is to solve is to provide a composition capable of preventing metabolic syndrome by increasing concentration of adiponectin, which is derived from a natural matter, causes fewer side effects and therefore is safe even when ingested for a prolonged period of time.

With the aim of solving the above-mentioned problem, intensive examinations have been carried out in the invention, and it was found as a result that it is possible to increase concentration of adiponectin in the living body by ingesting a powdered product or extract of a plant of the genus Salacia.

The invention includes the following constructions.

(1) An agent for increasing blood adiponectin quantity, which comprises:

a pulverized product or extract of a plant of the genus Salacia.

(2) The agent for increasing blood adiponectin quantity as described in (1) above,

wherein the plant of the genus Salacia is at least one species selected from the group consisting of Salacia oblonga, S. reticulata and S. prinoides.

(3) The agent for increasing blood adiponectin quantity as described in (1) or (2) above, which shows an activity as a sucrase 50% inhibition concentration (IC₅₀ value) of 50 μg/ml or more but 1,000 μg/ml or less.

(4) The agent for increasing blood adiponectin quantity as described in any one of (1) to (3) above, which shows an activity as a sucrase 50% inhibition concentration (IC₅₀ value) of 80 μg/ml or more but 600 μg/ml or less.

(5) The agent for increasing blood adiponectin quantity as described in any one of (1) to (4) above, which shows an activity as a sucrase 50% inhibition concentration (IC₅₀ value) of 100 μg/ml or more but 450 μg/ml or less.

(6) An agent for preventing or improving metabolic syndrome, which comprises:

the agent for increasing blood adiponectin quantity as described in any one of (1) to (5) above.

(7) A food or medicine, which comprises:

the agent for increasing blood adiponectin quantity as described in any one of (1) to (5) above.

More preferably, a method for alleviating a burden on the ingesting person can be provided, by containing calcium carbonate or silicon dioxide in a tablet, in an amount of 1% or more based on the total weight, which comprises a pulverized product or extract of a plant of the genus Salacia and has high sucrase inhibitory activity (small IC₅₀ value), and thereby preventing periodical discoloration of the tablet and reducing its necessary ingestion quantity. In addition, by the reduction of using amount, the range of applications from the viewpoint of production processing is also widened and the cost of the product can also be suppressed.

### BEST MODE FOR CARRYING OUT THE INVENTION

### Salacia

The agent of the invention for increasing blood adiponectin quantity contains a pulverized product or extract of a plant of the genus Salacia.

As the plant of the genus Salacia, Salacia reticulata, S. prinoides, S. oblonga and the like plants of the family Celastraceae, genus Salacia, can be used. Particularly, S. reticulata, which is also called kothalahimbutu, can be suitably used.

According to the invention, preferably an extract or pulverized product from at least one species selected from the group consisting of a trunk, a root skin and leaf of a plant of the genus Salacia can be used.

It is desirable that the leaf is used as a small piece or powder by pulverizing it. In addition, it can also be ingested as such.

The root skin and trunk can be used as powders. In addition, these can also be used for the extraction of an extract. The term extract as used herein means an extract of a plant of the genus Salacia. When used in the extraction of an extract, these may be used as such, or the extract can be extracted after making into small pieces or powders by pulverizing them.

According to the invention, the extract of a plant of the genus Salacia may be any one of the filtrate after extraction as such, or its concentrated or diluted state or in the form of its dried powder, or a mixture thereof.

The dry extract powder of the aforementioned extract can be used as such when used or by dissolving in an appropriate solvent. The aforementioned solvent may be any substance, with the proviso that it is a solvent which can be used at the time of extraction and does not exert a bad influence upon the human body even when it remained in the drug or foodstuff after preparation, and in general, water, an alcohol or a hydrous alcohol is preferably used. More preferably, hot water or ethanol or hydrous ethanol is used. Regarding the alcohol concentration of the aforementioned hydrous alcohol, those which have a concentration of from 30 to 90% by mass, preferably from 40 to 70% by mass, may be used. As the drying method, spray drying, freeze drying and the like can be exemplified, though not limited thereto. (In this specification, mass ratio is equal to weight ratio.)

It is preferable that the pulverized powder or powder-extracted extract powder of the root skin or trunk shows a weight loss on drying of 10% or less, more preferably a weight loss on drying of 8% or less, by the weight loss on drying test described in The Pharmacopoeia of Japan.

In addition, the extract or pulverized product of a plant of the genus Salacia can also be used in the form of a paste or powder by concentrating and drying the same. When the extract is made into a paste or powder by concentrating and drying it, a freeze drying method, a spray drying method and the like are used, though not limited thereto. The extract of a plant of the genus Salacia made into a paste or powder can be ingested as such, or a dried extract powder of the extract of a plant of the genus Salacia may be made into a food article, by adding to and mixing with a material containing water, tea, coffee, juice, alcohol and the like drinks, a cake and the like general food and the like as an inclusion composition, in an amount of 0.01% by mass or more of the inclusion composition. It can also be used for the purpose of an external use.

In the case of tablets or solid foodstuffs, the amount of the extract or pulverized product of a plant of the genus Salacia according to the invention is preferably 10 to 100% by mass, more preferably 20 to 100% by mass, based on the total amount of the agent for increasing blood adiponectin quantity. In the case of solutions or drinks, the amount of the extract or pulverized product of a plant of the genus Salacia according to the invention is preferably 0.01 to 20% by mass, more preferably 0.05 o 10% by mass, based on the total amount of the agent for increasing blood adiponectin quantity.

Particularly, the drinks are used as container-packed drinks. Since the appearance of a container-packed drink shows a large change in color tone when preserved for a prolonged period of time, it becomes unfit as goods. In the drinks, coloration gradually advances and the color tone is changed with the lapse of time.

By adding ascorbic acid, sodium ascorbate or the like antioxidant for the purpose of maintaining the color tone, it can exert further improved effect. Blending amount of the antioxidant is from about 0.03 to about 1.2% by mass, preferably from about 0.04 to about 1.0% by mass, more preferably from about 0.05 to about 0.8% by mass, based on the inclusion component.

<Other Contents>

It is desirable that the agent of the invention for increasing blood adiponectin quantity contains flavonoid in addition to the extract or pulverized product of a plant of the genus Salacia.

Flavonoid is a general term for the pigment components distributing in all plant organs, which is contained mainly in fruits and vegetables and is present particularly in the form of glycosides in skins of green and white vegetables and citrus fruits.

According to the invention, the flavonoid is a general term for the pigment components broadly distributing in plants, and particularly, it means flavan derivatives frequently contained in vegetables and fruits.

As the flavonoid, flavonols, isoflavones and catechins are preferable. Flavonols are known as polyphenols.

Flavonoid is a substance ingested into the body, but is generally hard to be absorbed. However, since flavonoid is effective even at a small amount and is a strong antioxidant, it is known that it suppresses the activity of carcinogens and has blood circulation accelerating action and anti-thrombus action.

According to the invention, flavonoid can be obtained from tea, grape, onion and the like respective origins. In this case, the origins mean those which are extracted from at least a part of an organism. For example, the above-mentioned method for preparing an extract of a plant of the genus Salacia can be employed for the extraction, and the form of the extract can also be the same as described in the above; for example, it may be any one of the filtrate after extraction as such, or its concentrated or diluted state or in the form of its dried powder, or a mixture thereof.

The tea extract containing catechins is prepared from a tea plant which is an evergreen tree belonging to the family Theaceae. As the tea plant, both of the assamica cultivated in India, Sri Lanka and Southeast Asia and Camellia sinensis cultivated in China and Japan can be used. In general, water, an alcohol or a hydrous alcohol is preferably used in the extraction. More preferably, hot water or ethanol or hydrous ethanol is used as the extraction solvent. Regarding the alcohol concentration of the aforementioned hydrous alcohol, those which having a concentration of from 30 to 90% by mass, preferably from 40 to 70% by mass, may be used. As the drying method, spray drying, freeze drying and the like can be exemplified, though not limited thereto.

Polyphenol, catechins and the like antioxidants are contained in the tea extract. It is desirable that catechin, epicatechin, gallocatechin, epigallocatechin, catechin gallate, epicatechin gallate, gallocatechin gallate or epigallocatechin gallate is contained therein, and it is particularly desirable that epigallocatechin gallate is contained therein.

It is preferable that the agent of the invention for increasing blood adiponectin quantity contains said tea extract in an amount of from 0.1 to 40% by mass, more preferably from 0.5 to 35% by mass, particularly preferably from 1.0 to 30% by mass:

Also, the flavonols as one of the flavonoid eliminate active oxygen and thereby show anti-oxidation actions such as suppression of arteriosclerosis and improvement of blood circulation. Among the flavonols, resveratrol as one of the polyphenol has been drawing attention as an antioxidant. Resveratrol is constituted from the stilbene backbone and contained in a large amount in the rind of grapes so that it is also contained in red wine produced from grapes.

It is desirable that the invention comprises a grape extract or grape wine concentrate which contains said flavonols as a component.

It is preferable that the agent of the invention for increasing blood adiponectin quantity contains the grape extract in an amount of from 0.1 to 30% by mass, more preferably from 0.1 to 10% by mass.

It has been revealed that resveratrol has the actions to burn fat, to prevent a blood vessel system disease, arteriosclerosis, to exert anti-cancer action and to prevent shortening of DNA caused by cell division, and has the effect to prolong life of cells similar to the case of carrying out calorie control, so that it has the excellent effect as a material for preventing life style-related diseases.

The resveratrol content in the agent of the invention for increasing blood adiponectin quantity is preferably from 0.0001 to 5.00% by mass, more preferably from 0.001 to 2.00% by mass.

In addition, among the flavonols, quercetin as a polyphenol has been drawing attention as an antioxidant. Quercetin has the flavan structure and is contained in a large amount in onion skins.

Vitamin C absorption support, anti-oxidation action, immune action and the like physiological actions of quercetin have been reported, and it has been revealed that it is effective in suppressing fat absorption and it has been revealed also that it has the excellent effect as a material for preventing life style-related diseases.

The quercetin content in the agent of the invention for increasing blood adiponectin quantity is preferably from 0.001 to 15% by mass, more preferably from 0.05 to 10% by mass, further preferably from 0.1 to 5.0% by mass.

It is preferable that the agent of the invention for increasing blood adiponectin quantity contains catechin in an amount of from 1 to 50% by mass. As the catechin, a green tea-derived one or the like is particularly desirable.

In addition, it is preferable that the agent of the invention for increasing blood adiponectin quantity contains a polyphenol having lipase activity inhibitory effect in an amount of from 2 to 80% by mass. As the polyphenol having lipase activity inhibitory effect, those derived from Oolong tea, derived from grape, derived from apple, derived from Lychee, derived from pine bark, derived from kanka and the like are particularly desirable.

<Shape>

The agent of the invention for increasing blood adiponectin quantity can be used as both of food and pharmaceutical preparations. In addition, the agent of the invention for increasing blood adiponectin quantity can take powder preparations, tablets, solutions, capsule preparations and the like various shapes.

According to the invention, in order to improve periodical discoloration of the extract powder extracted from the plant of the genus Salacia, it is desirable to contain 1% by mass or more of calcium carbonate or silicon dioxide as a desiccant in forming tablets or capsules.

Further, a low moisture absorption material, a moisture absorbent, an antioxidant and the like which are applicable as a foodstuff or food additive agent can be used. Preferably, cellulose, crystalline cellulose, cellulose powder, microcrystalline cellulose, lactose, an oligosaccharide, a sugar alcohol, trehalose, magnesium stearate, calcium stearate or the like is used as the low moisture absorption material. As the moisture absorbent, silicates, magnesium carbonate, a ferrocyanide, polysaccharides or the like are used. More preferably, crystalline cellulose, microcrystalline cellulose or lactose is used as the low moisture absorption material. As the antioxidant, ascorbic acid, sodium ascorbate or the like is used.

A compound necessary for forming into the powder, solid preparation or liquid preparation of the invention, and the like may be optionally contained. As examples of such a compound, erythritol, maltitol, hydroxypropylcellulose, kaolin, talc and the like can be cited.

According to the invention, conventionally known methods and conventionally known materials can be applied to the preparation for obtaining powder preparations, tablets or solutions, granulation of capsule inclusion matter for forming capsule preparations, capsulation, capsule material and the like.

The capsule preparation of the invention may be in a hard capsule, soft capsule, seamless capsule, microcapsule or the like shape, and is characterized in that the capsule shell is constructed by at least one or two or more species selected from pig skin gelatin, pig bone gelatin, fish gelatin and a natural hydrophilic polymer. A capsule shell of pig skin gelatin or fish gelatin is particularly desirable.

These capsule shells can be produced by a conventionally known method. In this case, the term “constructed by pig skin gelatin, pig bone gelatin, fish gelatin or a natural hydrophilic polymer” means that total amount of the pig skin gelatin, pig bone gelatin, fish gelatin and natural hydrophilic polymer is 30% by mass or more, preferably 40% by mass or more, more preferably 50% by mass or more, particularly preferably 60% by mass or more, based on the total mass of capsule shell.

In addition, in order to avoid its contact with air from the viewpoint of preventing oxidation, it is desirable to pack the above-mentioned composition in a packing bag or packing container.

Preferably, a tablet containing an extract of a plant of the genus Salacia, which has high sucrase inhibitory activity, is used. By this, its effect can be obtained with a small amount when ingested as a food or the like and the burden for the ingesting person therefore is alleviated. In addition, when a product is prepared, its stability is increased, which also becomes useful from the production and processing points of view.

It is desirable that the agent of the invention for increasing blood adiponectin quantity has a sucrase 50% inhibition concentration (IC₅₀ value) of 50 μg/ml or more but 1,000 μg/ml or less. When the inhibition activity is within this range, the glucose absorption inhibitory action from the digestive tracts works and the expected effects, can be obtained, and a feeling of abdominal swelling and generation of gas can be prevented. The sucrase 50% inhibition concentration is preferably 80 μg/ml or more but 600 μg/ml or less, more preferably 100 μg/ml or more but 450 μg/ml or less.

The sucrase 50% inhibition concentration (IC₅₀ value) is measured by the following method.

### [Test Method 1] Measurement of Sucrase IC₅₀ Value

Preparation of sample solution: A 2 mg portion of a sample is weighed and put into a tube and thoroughly suspended in 2 ml of water added thereto, thereby preparing a sample solution having a concentration of 1 mg/ml. This is diluted with water to respective concentrations of 0, 50, 100, 250 and 500 μg/ml.

Preparation of substrate liquid: Sucrose is dissolved in 0.2 M maleate buffer (pH 6.0) to a sucrose concentration of 100 mM, and this is used as the substrate liquid.

Preparation of crude enzyme liquid: A 1 g portion of intestinal acetone powder rat (mfd. by SIGMA) is suspended in 10 ml of physiological saline and then centrifuged (3,000 rpm, 4° C., 5 min). The thus obtained supernatant is separated and used as the crude enzyme liquid.

A 400 μl portion of the substrate liquid is added to 500 μl of each of the aforementioned sample solution having respective concentrations and preliminarily heated at 37° C. for 5 minutes in a water bath. A 100 μl portion of the crude enzyme liquid is added to each of them and allowed to undergo the reaction at 37° C. for 60 minutes. After completion of the reaction, the reaction is terminated by deactivating the enzyme through heating at 95° C. for 2 minutes. Determination of concentration of the thus formed glucose is carried out using a commercially available kit for mutarotase glucose oxidase method (Glucose CII Test Wako, mfd. by Wako Pure Chemical Industries).

Preparation of blank: A 200 μl portion of the substrate liquid and 50 μl of the crude enzyme liquid are added to 250 μl of each of the aforementioned sample solution having respective concentrations and immediately heated at 95° C. for 2 minutes to effect thermal deactivation of the enzyme, to be used as blank data.

By preparing a calibration curve from the thus obtained values, the concentration which inhibits 50% of the enzyme activity (IC₅₀ value) is calculated.

The ingestion amount of the agent for increasing blood adiponectin quantity is, in terms of an amount of the extract or pulverized product of a plant of the genus Salacia per day, preferably 5 to 5,000 mg, more preferably 10 to 2,000 mg.

The agent for increasing blood adiponectin quantity is ingested preferably once to six times a day, more preferably three times a day in parts.

The ingestion is preferably conducted before meal or after meal, and the ingestion within an hour before meal to two hours after meal is preferred, and the ingestion within 30 minutes before meal to an hour after meal is more preferred.

### EXAMPLES

The following describes the invention using examples, but the invention is not limited to the following examples.

### Example 1

Root and trunk parts of Salacia reticulata and Salacia oblonga were pulverized and then subjected to a hot water extraction step at 95° C., and the thus obtained liquid was spray-dried to obtain a Salacia extract powder 1.

In addition, root and trunk parts of Salacia prinoides were pulverized and then subjected to an extraction step by ethanol-water (ethanol 30% by volume) at 40° C., and the thus obtained liquid was mixed with dextrin and spray-dried to obtain a Salacia extract powder 2 (dextrin content 30% by weight).

Using these Salacia extract powders, powders having the formulations of the samples 1 to 8 shown in Table I were prepared and their sucrase IC₅₀ values were measured by the method described in the “Test method 1”.

Using these, the formulation components shown in the following Table 1 were subjected to tablet making to prepare the samples 1 to 8.

TABLE 1
Salacia formulation example and sucrase IC₅₀ value

| | Salacia | Salacia | | Sucrose | |
|---|---|---|---|---|---|
| | extract | extract | Crystalline | IC₅₀ | |
| | powder 1 | powder 2 | cellulose | value | Examples |
| Sample 1 | 0 mg | 0 mg | 250 mg | >2000 | Comparative |
| Sample 2 | 10 mg | 0 mg | 240 mg | 1750 | Inventive |
| Sample 3 | 20 mg | 0 mg | 230 mg | 910 | Inventive |
| Sample 4 | 100 mg | 0 mg | 150 mg | 182 | Inventive |
| Sample 5 | 220 mg | 0 mg | 30 mg | 53 | Inventive |
| Sample 6 | 0 mg | 10 mg | 240 mg | 890 | Inventive |
| Sample 7 | 0 mg | 20 mg | 130 mg | 460 | Inventive |
| Sample 8 | 0 mg | 100 mg | 50 mg | 90 | Inventive |

After carrying out sufficient informed consensus for the persons to be tested, each group of 5 healthy adults who desired the participation by free will was allowed to orally ingest one tablet of each of the samples 1 to 8 respectively within 30 minutes after meal every day (three tablets a day), and this was repeated for 90 days.

By collecting blood samples before commencement of the ingestion, after 60 days of the ingestion and on the next day of the completion of the ingestion, amounts of adiponectin in the blood samples were measured and compared. The blood adiponectin was measured by an enzyme immunoassay which uses an antibody prepared using a recombinant adiponectin (a kit manufactured by Otsuka Pharmaceutical was used).

Average amount of blood adiponectin in each sample ingestion group is shown in Table 2. The amount of adiponectin was shown by relative values when the amount before ingestion is regarded as 100.

TABLE 2
Amount of blood adiponectin after 60
days or 90 days of sample ingestion

| | Amount of adiponectin | Amount of adiponectin | |
|---|---|---|---|
| | after 60 days of | after 90 days of | |
| | ingestion (μg/dl) | ingestion (μg/dl) | Examples |
| Sample 1 | 97 | 98 | Comparative |
| Sample 2 | 103 | 105 | Inventive |
| Sample 3 | 106 | 114 | Inventive |
| Sample 4 | 122 | 127 | Inventive |
| Sample 5 | 120 | 135 | Inventive |
| Sample 6 | 108 | 110 | Inventive |
| Sample 7 | 110 | 118 | Inventive |
| Sample 8 | 118 | 122 | Inventive |

It was revealed that amount of the blood adiponectin is increased by the ingestion of the samples of the invention.

This effect was most significant by a sample having a sucrase 50% inhibition concentration (IC₅₀ value) of 50 μg/ml or more but 1,000 μg/ml or less.

In addition, since it is conventionally known that adiponectin prevents metabolic syndrome, it was revealed that the samples of the invention are possessed of the effects to prevent and improve metabolic syndrome.

### Example 2

### A Tablet Using Salacia Extract Powder

By preparing a tablet using the formulation shown in Table 3, a supplement to which shellac coating was applied was prepared. The sucrase 50% inhibition concentration (IC₅₀ value) of this example was 210 μg/ml.

The amount of the tablet was 250 mg per tablet, and the tablet was ingested three times a day before every meal.

TABLE 3
Tablet formulation example using the
Salacia extract powder of the invention

| Raw material name | Blending amount (wt %) |
|---|---|
| Salacia extract powder 1 | 25.0 |
| Red wine polyphenol | 10.0 |
| Onion outer skin extract powder | 6.0 |
| Green tea extract | 15.0 |
| Hematococcus algal pigment | 1.0 |
| Chrome yeast | 4.0 |
| Carnitine | 10.0 |
| Crystalline cellulose | 23.0 |
| Sucrose fatty acid ester | 2.0 |
| Lactose | 1.0 |
| Calcium carbonate | 1.0 |
| Atomized silicon dioxide | 2.0 |

The effects shown by Example 1 was obtained also by the ingestion of the tablet of this formulation.

In addition, belly size became neat, the body became lighter, hangover became hard to occur and the like reports were obtained from the ingesting persons to be tested.

### Example 3

### A Drink Using Salacia Extract Powder 2

A drink by the formulation shown in Table 4 was prepared. The sucrase 50% inhibition concentration (IC₅₀ value) of this example was 900 μg/ml.

TABLE 4
Drink formulation example using the
Salacia extract powder of the invention

| Raw material name | Blending amount (g) |
|---|---|
| Salacia extract powder 2 | 2.0 |
| Fructose glucose syrup | 120.0 |
| Vitamin C (L-ascorbic acid) | 10.0 |
| Citric acid | 10.0 |
| Orange perfume | 3.0 |
| Water | 855.0 |
| Total | 1000.0 |

A drinking liquid was prepared by mixing and dissolving the components of Table 3. This was filled in 50 cc portions into bottles and heat-sterilized at 85° C. for 10 minutes, and this was cooled to room temperature and used as a drink. A bottle of the drink (50 cc) was ingested three times a day before every meal.

The effects shown by Example 1 was obtained also by the ingestion of the drink of this formulation.

In addition, belly size became neat, the body became lighter, hangover became hard to occur and the like reports were obtained from the ingesting persons to be tested.

### INDUSTRIAL APPLICABILITY

By the invention, it becomes possible to increase concentration of adiponectin in the living body. By this, an agent for increasing blood adiponectin quantity capable of preventing metabolic syndrome, which is derived from a natural matter, causes fewer side effects and therefore is safe even when ingested for a prolonged period of time, is provided.

The entire disclosure of each and every foreign patent application from which the benefit of foreign priority has been claimed in the present application is incorporated herein by reference, as if fully set forth.

1. An agent for increasing blood adiponectin quantity, which comprises:
a pulverized product or extract of a plant of the genus Salacia.

2. The agent for increasing blood adiponectin quantity according to claim 1,
wherein the plant of the genus Salacia is at least one species selected from the group consisting of Salacia oblonga, S. reticulata and S. prinoides.

3. The agent for increasing blood adiponectin quantity according to claim 1, which shows an activity as a sucrase 50% inhibition concentration (IC₅₀ value) of 50 μg/ml or more but 1,000 μg/ml or less.

4. The agent for increasing blood adiponectin quantity according to claim 1, which shows an activity as a sucrase 50% inhibition concentration (IC₅₀ value) of 80 μg/ml or more but 600 μg/ml or less.

5. The agent for increasing blood adiponectin quantity according to claim 1, which shows an activity as a sucrase 50% inhibition concentration (IC₅₀ value) of 100 μg/ml or more but 450 μg/ml or less.

6. An agent for preventing or improving metabolic syndrome, which comprises:
the agent for increasing blood adiponectin quantity according to claim 1.

7. A food or medicine, which comprises:
the agent for increasing blood adiponectin quantity according to claim 1.
